(19) United States
(12) Patent Application Publication
(10) Pub. No.: US 2024/0122266 A1
(43) Pub. Date: Apr. 18, 2024

(54) Aerosol Generation System and Computer-Implemented Method

(71) Applicant: JT International SA, Geneva (CH)

(72) Inventors: Stéphane Hedarchet, Geneva (CH); Burkhard Dettmar, Trier (DE)

(73) Assignee: JT International SA, Geneva (CH)

(21) Appl. No.: 18/277,659

(22) PCT Filed: Feb. 18, 2022
(86) PCT No.: PCT/EP2022/054070

§ 371 Date: Aug. 17, 2023

(30) Foreign Application Priority Data
Feb. 19, 2021 (EP) 21158289.5

(51) Int. Cl. A24F 40/53; A24F 40/30; A24F 40/51; A24F 40/65

(52) U.S. Cl. CPC A24F 40/53; A24F 40/30; A24F 40/51; A24F 40/65; A24F 40/10

(57) ABSTRACT

An aerosol generation system includes an aerosol generation device configured to receive each of a plurality of aerosol generation substrates and to generate a respective aerosol to be inhaled by a user; a feedback interface for receiving user response data in reaction to inhaling the generated aerosol; and a preference identification system. The preference identification system being configured to: for each of the plurality of aerosol generation substrates, obtain user response data from the feedback interface; and identify a preferred aerosol for the user based on the user response data.

### TECHNICAL FIELD

The present disclosure relates to aerosol generation devices in which an aerosol generation substrate is aerosolized for inhalation by a user of the device. The present disclosure also relates to methods for controlling aerosol generation by aerosol generation devices.

### BACKGROUND

The popularity and use of reduced-risk or modified-risk devices (also known as vaporisers) has grown rapidly in the past few years as an aid to assist habitual smokers wishing to quit smoking traditional tobacco products such as cigarettes, cigars, cigarillos, and rolling tobacco. Various devices and systems are available that heat or warm aerosolisable substances as opposed to burning tobacco in conventional tobacco products.

Different users of such devices have different preferences for the composition of the inhaled aerosol. For example, different users may desire different nicotine concentrations or different flavours. It is known to provide a range of aerosol generation substrates to be used as alternatives in an aerosol generation device. In this way, a range of aerosol compositions can be offered for a single aerosol generation device.

However, in known systems, the user is required to identify their preferred composition of the inhaled aerosol. It would be desirable to provide an aerosol generation system capable of assisting the user in identifying their preferred aerosol composition.

### SUMMARY

According to a first aspect, there is provided an aerosol generation system comprising:
an aerosol generation device configured to receive each of a plurality of aerosol generation substrates and to generate the respective aerosol to be inhaled by a user;
a feedback interface for receiving user response data of the user in reaction to inhaling the generated aerosol; and
a preference identification system configured to: for each of the plurality of aerosol generation substrates, obtain user response data from the feedback interface; and identify a preferred aerosol for the user based on the user response data.

User response data may be data of an involuntary response and/or data of a voluntary response. More specifically, an “involuntary response” is a reaction of the user that is not consciously chosen and that can be measured without conscious user participation. In contrast, a “voluntary response” is deliberate user feedback, for example by pressing a button, or by entering a rating in a graphical user interface.

Optionally, the preference identification system is configured to obtain initial user response data from the feedback interface for each of a sample plurality of aerosol generation substrates, the sample plurality of aerosol generation substrates being provided in a pack; and identify an initial preferred aerosol for the user based on the initial user response data. In this way, the user is guided through trialling an initial set of aerosol generation substrates, in order to select a preferred aerosol and to identify a corresponding aerosol generation substrate which the user may advantageously use in future.

Furthermore, the respective aerosols generated from the sample plurality of aerosol generation substrates are optionally distributed across a sensory range of different compositions. For example, the sensory range may comprise a range of strengths of each of one or more aerosol components. By providing a sampling pack comprising a range of aerosol generation substrates, the chance of the user successfully identifying their most preferred aerosol among an available range is improved.

Optionally, the preference identification system is further configured to: obtain subsequent user response data from the feedback interface for one or more subsequent aerosol generation substrates; identify an updated preferred aerosol for the user based on the subsequent user response data and the initial preferred aerosol. In this way, the aerosol generation system may iteratively improve its identification of a preferred aerosol for the user.

Optionally, the preference identification system is configured to: identify a plurality of candidate preferred aerosols for the user based on the user response data; and present the plurality of candidate preferred aerosols in a user interface. By offering the user a set of candidate preferred aerosols, the system further improves the chance of the user successfully and simply identifying their most preferred aerosol.

Furthermore, the preference identification system is optionally configured to receive a user selection of the preferred aerosol from among the candidate preferred aerosols. By enabling a user selection among candidate options, the user is provided with a simple interface for selecting an aerosol while also increasing the chance that the available options (candidate aerosols) include an aerosol which is particularly preferable for the user.

Optionally, the aerosol generation device is configured to receive at least two of the plurality of aerosol generation substrates simultaneously, and to control which of the received aerosol generation substrates is used to generate an aerosol. In this way, the aerosol generation device can automatically switch between generating at least two different aerosols, improving user convenience when trialling a range of aerosols.

Furthermore, the aerosol generation device is optionally configured to receive an indication of the preferred aerosol, and to control which of the received aerosol generation substrates is used to generate an aerosol, based on the preferred aerosol. In this way, the aerosol generation system can automatically switch to generating the identified preferred aerosol.

Additionally, the aerosol generation device optionally comprises an aerosol generation chamber and a substrate mixing element configured to mix a plurality of the simultaneously-received aerosol generation substrates and supply a mixed aerosol generation substrate to the heating chamber. In this way, the aerosol generation device can produce new aerosol generation substrates, and the user can identify a preferred aerosol to a greater degree of precision, without being limited by a predetermined set of possible aerosol generation substrates.

Optionally, the preference identification system is configured to identify the preferred aerosol based on the user response data and based on a predetermined model relating user response data to aerosol preference. By using a predetermined model, the user's preferred aerosol can be more accurately identified with less data from the specific user.

Optionally, the preference identification system is configured to: obtain user response data for each of a plurality of training users; and train the model to predict a preferred aerosol based on user response data for the plurality of training users. The trained model can subsequently be used to identify a user's preferred aerosol with less data from the specific user.

Optionally, the feedback interface comprises a sensor for measuring an involuntary response of the user, and the user response data comprises an indication of the involuntary response. By using an involuntary response, accuracy and convenience of the system is improved, because user feedback is automatically collected using involuntary responses, and the system does not rely on the user to accurately indicate or quantify aerosol preference.

Optionally, the sensor is configured to measure a heart rate, a blood pressure, a skin temperature or a galvanic skin response. Any of these are responses which the user has no direct control over and which can be used to infer a level of relaxation or stress, or to otherwise infer a quality of user experience.

Optionally, the sensor is arranged on an exterior surface of a housing of the aerosol generation device. With this arrangement, the sensor can be near to or directly in contact with the user when they are holding the aerosol generation device, and an involuntary response can be measured without requiring any additional action by the user beyond using the aerosol generation device.

Optionally, the feedback interface comprises a voluntary response interface for receiving a voluntary response from the user in reaction to inhaling the generated aerosol, and the user response data comprises an indication of the voluntary response. By obtaining voluntary responses, the system can improve accuracy of identifying a preferred aerosol, by calibrating the interpretation of involuntary responses. Alternatively, where voluntary responses are received, the system can be simplified by not measuring involuntary responses,

Optionally, the feedback interface comprises an air flow sensor arranged to measure aerosol inhalation at the aerosol generation device when the user is inhaling the generated aerosol, and the user response data comprises an indication of the aerosol inhalation. By measuring aerosol inhalation, the preference identification system can additionally identify the preferred aerosol based on a duration of inhalation, or a number of times that the aerosol is inhaled, as a measure of the user's appreciation of the aerosol, and thereby more accurately identify the preferred aerosol.

Optionally, the aerosol generation device comprises a substrate identification means for identifying a received aerosol generation substrate, and the user response data comprises an indication of the received aerosol generation substrate. By providing a substrate identification means, the user is not required to manually tell the preference identification system which aerosol generation substrate they have used, and accuracy and user convenience are improved.

Optionally, the preference identification system is further configured to determine a health characteristic of the user based on the involuntary response data, wherein identifying the preferred aerosol comprises allowing or blocking an aerosol generation substrate based on the health characteristic. By controlling the available aerosols based on a determined health characteristic, an aerosol that is more likely to be suitable for the user can be chosen.

According to a second aspect, the present disclosure provides a pack comprising a sample plurality of aerosol generation substrates, each for generating a different respective aerosol in an aerosol generation system, wherein the different respective aerosols are distributed across a sensory range of different compositions such that a preferred aerosol can be identified based on user response data obtained in reaction to inhaling each of the aerosols.

According to a third aspect, the present disclosure provides a kit comprising a pack according to the second aspect and an aerosol generation device configured to receive each of the plurality of aerosol generation substrates and to generate the respective aerosol to be inhaled by a user, the kit being for use in an aerosol generation system according to the first aspect.

According to a fourth aspect, the present disclosure provides methods and computer programs for operating an aerosol generation system according to the first aspect. The computer programs may be stored as instructions in a memory storage medium or in a data signal, or may be hard-coded in circuitry. In particular, the preference identification system may be implemented as software to be executed on a computing system that is located in, or is in communication with, the aerosol generation device and the feedback interface. More specifically, the present disclosure provides a method of controlling an aerosol generation system, the aerosol generation system comprising: an aerosol generation device configured to receive an aerosol generation substrate and to generate a respective aerosol to be inhaled by a user; and a feedback interface for receiving user response data of the user in reaction to inhaling the generated aerosol, the method comprising: for each of a plurality of aerosol generation substrates, using the aerosol generation device to generate a respective aerosol, and obtaining user response data from the feedback interface; and identifying a preferred aerosol for the user based on the user response data.

### BRIEF DESCRIPTION OF THE DRAWINGS

FIGS. 1A and 1B are block diagrams schematically illustrating an aerosol generation device and a pack according to the invention;

FIG. 2 is a block diagram schematically illustrating an aerosol generation system according to an embodiment;

FIG. 3 is a flow chart schematically illustrating a computer-implemented method for controlling the aerosol generation system;

FIG. 4 is a flow chart schematically illustrating optional features of a method for controlling the aerosol generation system;

FIG. 5A is a flow chart schematically illustrating further optional features of a method for controlling the aerosol generation system;

FIG. 5B is a graphical depiction of a trained model for identifying a preferred aerosol;

FIG. 5C is a flow chart schematically illustrating a method for training the model; and

FIG. 6 is a block diagram schematically illustrating an alternative aerosol generation device according to the invention.

### DETAILED DESCRIPTION

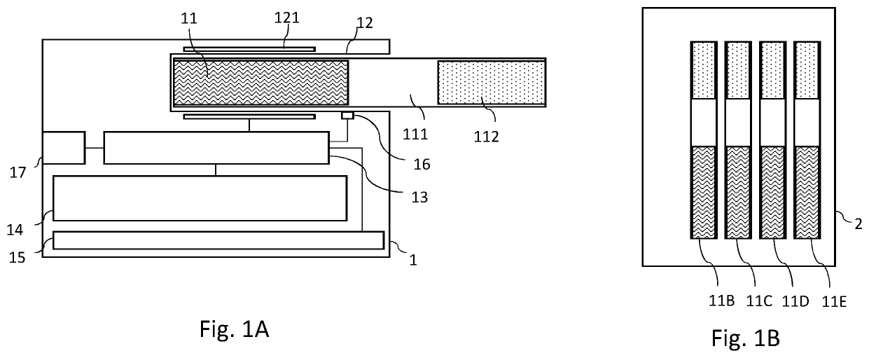

FIG. 1A schematically illustrates an aerosol generation device for use in an aerosol generation system according to the invention.

The aerosol generation device 1 comprises an aerosol generation chamber 12 and control circuitry 13.

The aerosol generation chamber 12 is configured to receive an aerosol generation substrate 11. The aerosol generation chamber 12 may, for example, take the form of a pot which has an opening at one end to allow the aerosol generation substrate 11 to be inserted and removed.

The aerosol generation substrate 11 may for example take the form of a solid package which can be easily inserted into and removed from the aerosol generation device. Alternatively, the aerosol generation substrate may be a loose material or a liquid. As one example, the substrate may comprise tobacco. The tobacco may for example comprise randomly oriented tobacco strands containing tobacco powder and an aerosol former.

The aerosol generation device 1 is configured to generate an aerosol from the aerosol generation substrate 11, so that the aerosol can be inhaled by a user. In the embodiment of FIG. 1, the aerosol is generated by heating the aerosol generation substrate 11 using a heater 121. The heater may be arranged on at least one side of the aerosol generation chamber 12, for example in the form of a thin-film heater or a ceramic heater, or may be located in the aerosol generation chamber 12, for example in the form of a blade heater. The aerosol may instead be generated by other means, such as an atomizer or a nebulizer.

The aerosol generation device 1 may further comprise a mouthpiece for a user to obtain aerosol generated in the aerosol generation chamber 12. Alternatively, as shown in FIG. 1, the aerosol generation substrate 11 may be provided as part of a package that includes a disposable mouthpiece. More specifically, in the example of FIG. 1, the aerosol generation substrate 11 is provided in a cigarette-style package in which a wrapper 111 encloses the substrate 11 and a filter 112.

The control circuitry 13 is configured to control the heating element 121 and thereby to control aerosol generation. The control circuitry 13 is configured to draw power from an electrical power source in order to control and drive aerosol generation. The electrical power source is preferably included in the aerosol generation device, for example taking the form of battery 14. Alternatively, the control circuitry 13 may be provided with an external power supply.

Referring now to FIG. 1B, the aerosol generation substrate 11 may be provided as part of a pack 2 comprising a sample plurality of aerosol generation substrates 11B to 11E. Each of the sample plurality of aerosol generation substrates 11B to 11E may be a different aerosol generation substrate which generates a different aerosol.

The respective aerosols generated from the sample plurality of aerosol generation substrates may be distributed across a sensory range of different compositions. The user will react differently to each aerosol, and thus the range of aerosol generation substrates will produce a range of different user response data which can be used to identify a preferred aerosol.

More specifically, the aerosol generation substrates may have a range of strengths of each of one or more aerosol components. For example, different substrates may have different strengths of nicotine or different strengths of flavours such as mint.

A pack 2 as shown in FIG. 1B may be provided together with an aerosol generation device 1 as shown in FIG. 1A, as a kit for use in an aerosol generation system.

Referring again to FIG. 1A, in this embodiment, the aerosol generation device 1 also comprises a sensor 15 that is configured to measure the involuntary response of the user, as user response data in reaction to inhaling a generated aerosol. Involuntary responses may be indicative of, for example, arousal or positive/negative affect. Involuntary responses which may be measured by the sensor 15 include heart rate, blood pressure, or galvanic skin response (where galvanic skin response is correlated with sweating) as indicators of arousal, or skin temperature as an indicator for affect. Involuntary responses may, for example, be sampled at a sampling rate and averaged or normalised in order to reduce noise and base-line effects.

The sensor 15 may, for example, be arranged on an exterior surface of a housing of the aerosol generation device. In this position, the sensor 16 can contact a hand of the user when they are holding the aerosol generation device and measure a response through the finger tips. For example, the housing-located sensor may be particularly suitable for measuring galvanic skin response and skin temperature.

Additionally, in the embodiment shown in FIG. 1, the aerosol generation device 1 comprises an air flow sensor 16 arranged to measure aerosol inhalation at the aerosol generation device 1 when the user is inhaling the generated aerosol. This measured inhalation may be used as a part of the user response data. The air flow sensor 16 may be located in or by a mouthpiece, or may be located elsewhere in the aerosol generation chamber 12. The air flow sensor 16 is configured to measure aerosol inhalation by a user. This measured aerosol inhalation can comprise a length or volume of continuous inhalation, a frequency or number of instances of inhalation, and/or a strength or pressure drop of the inhalation. The measured aerosol inhalation can be used in identifying a preferred aerosol. For example, a large number or rate of puffs may indicate an enjoyable or soothing aerosol, while a low number or rate of puffs may suggest an aerosol which invokes a longer-lasting response.

More generally, the aerosol generation system of the invention (which includes the aerosol generation device 1) comprises a feedback interface for receiving user response data of the user in reaction to inhaling the generated aerosol. The feedback interface may include the sensor 15 and/or the air flow sensor 16 and/or one or more further sensors collecting involuntary response data of a user in reaction to inhaling a generated aerosol.

The feedback interface may be separate from the aerosol generation device, and may comprise multiple interfaces which are each either part of or separate from the aerosol generation device.

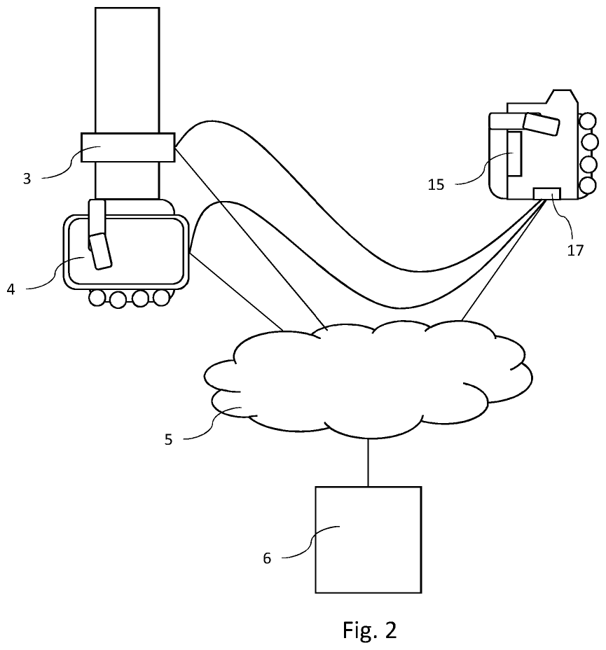

FIG. 2 is a block diagram schematically illustrating an aerosol generation system according to an embodiment.

Referring to FIG. 2, the feedback interface may comprise a sensor in a wearable device 3 such as a smart watch. A wearable sensor may be particularly suitable for measuring an involuntary response through the skin, such as a heart rate or blood pressure, where the response is not so easy to measure through the finger tips.

As additionally shown in FIG. 2, the feedback interface may comprise a sensor in a smart device with a display, such as a smart phone 4. Either of the wearable device 3 and the smart phone 4 may communicate with the aerosol generation device 1 via a wireless communication module 17 (also shown in FIG. 1), and optionally also via a network 5 such as a wireless local area network (WLAN).

In addition to sensors configured to obtain involuntary response data, the feedback interface may receive user response data in the form of voluntary response data. For example, the smart phone 4 may display a prompt for the user to give feedback. The user feedback may take the form of an affective liking score, such as a rating of the aerosol, e.g. on a scale of 1 to 5 stars, or any other rating. The user feedback may also take the form of a comparison between a current aerosol and a previously-used aerosol.

As will be discussed further below, the aerosol generation system further comprises a preference identification system. The preference identification may be implemented in the control circuitry 13, but is more preferably at least partly implemented in a remote processing unit 6. The remote processing unit 6 may be, for example, a physical server or cloud server. The remote processing unit 6 may also, for example, be a system comprising more than one physical server or cloud server.

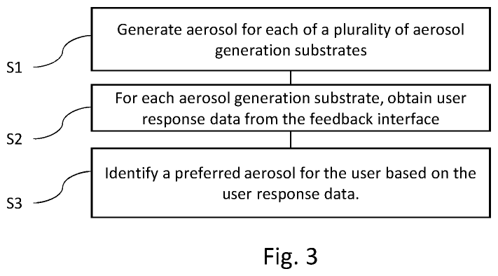

A method for operating the control circuitry 13 is schematically illustrated in FIG. 3 using a flow chart. This method may be stored as computer program instructions in a memory within control circuitry 13 and/or smartphone 4 and/or remote processing unit 6. Instructions defining the method may be installed or updated using a communication signal, such as via a network connection, or using a computer-readable storage medium. Alternatively, the control circuitry 13, smartphone 4 and/or remote processing unit 6 may comprise an application specific integrated chip (ASIC) or other custom hardware configured to perform the method.

Referring to FIG. 3, at step S1, the control circuitry 13 controls the aerosol generation chamber 12 to generate an aerosol according to each of a plurality of different aerosol generation substrates.

Generation of each different aerosol may comprise generating an aerosol from a different aerosol generation substrate, and/or generating an aerosol at a different strength of aerosol generation by the heating element 121.

For example, step S1 may comprise the user swapping each of the substrates 11B to 11E of a pack 2 (FIG. 1B) into the aerosol generation device 1, and the aerosol generation device 1 generating a respective aerosol from each substrate.

The different aerosols preferably comprise a range of predetermined aerosols that are known to be preferred by individual users. Alternatively, the aerosol preferences may be constrained to a large but finite number of possible aerosol preferences.

At step S2, the preference identification system obtains user response data for a user of the aerosol generation device. Step S2 is performed for each of the different aerosols of step S1, and thus step S2 is performed in parallel with step S1.

Once steps S1 and S2 have been performed to generate a plurality of different aerosols and to obtain user response data for each setting, at step S3, the control circuitry 13 identifies a preferred aerosol for the user based on the user response data.

For example, at step S3, the control circuitry 13 may identify an aerosol associated with an involuntary response indicating greatest relaxation of the user. In a more specific example, where the sensor 15 measures a heart rate, an aerosol that prompted an involuntary response comprising the lowest heart rate may be identified as the preferred aerosol. The control circuitry 13 may then store the identified aerosol in a memory as a default aerosol.

Steps S1 to S3 may be performed iteratively to identify the user's preferred aerosol with increased precision. For example, a first iteration of step S1 may comprise generating an aerosol from each of a plurality of aerosol generation substrates 11 in a pack 2. The first iteration may identify which of the pure aerosol generation substrates gives an aerosol that is most preferable for the user, as an initial preferred aerosol. Subsequent iterations of steps S1 to S3 may then obtain subsequent user response data from the feedback interface for one or more subsequent aerosol generation substrates, and identify an updated preferred aerosol based on the subsequent user response data and the initial preferred aerosol.

The preference identification system may be software directly implemented by the control circuitry 13 in the aerosol generation device 1. Alternatively, the functions of the preference identification system may be performed at least in part at the remote processing unit 5. For example, the preference identification system may perform at least part of step S3 by analysing raw data obtained by the control circuitry 13 in order to identify a preferred aerosol. The preference identification system may perform complex learning analyses such as by following a Design of Experiment (DoE) approach, and may generate a multidimensional map of involuntary responses to aerosols. Such a map may be based on a Tucker-1 Principal Component Analysis (PCA).

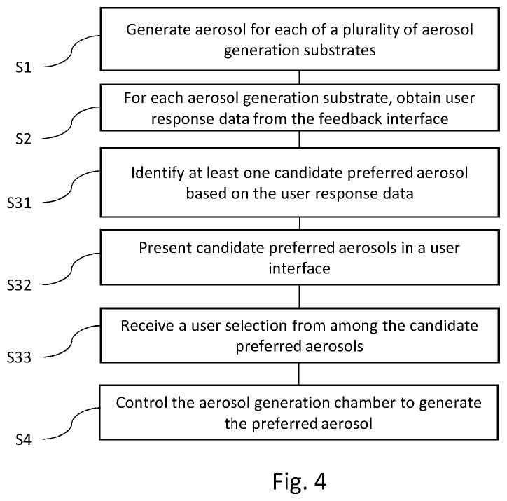

FIG. 4 is a schematic flow chart illustrating optional features of the method shown in FIG. 3. Steps S1 and S2 are identical to the above-described method of FIG. 3, and will not be described again.

Referring to FIG. 4, the user is presented with a choice of aerosols based on previously collected user response data.

At step S31, the control circuitry 13 or the remote processing unit 5 identifies at least one, and preferably several, candidate preferred aerosols. In this method it is not necessary to automatically select only one preferred aerosol, and multiple aerosols can be identified as candidates.

At step S32, the candidate preferred aerosol(s) are presented in a user interface. For example, the candidates may be presented on a user interface of the aerosol generation device 1 or the smart device 3.

For example, the candidate preferred aerosols may be presented as a sensory map, from which the user can select a preferred aerosol.

At step S33, the user interface receives a user selection of a preferred aerosol that is to be used. The selected aerosol is then communicated directly or indirectly to the control circuitry 13 in the aerosol generation device 1.

At step S4, the control circuitry 13 controls the aerosol generation chamber 12 to generate the preferred aerosol as identified in step S3 (steps S31 to S33). Step S4 may be repeated using the preferred aerosol, as often as the user wishes.

The modifications to step S3 and the addition of step S4 are independent possible modifications of the method of FIG. 3. In other words, any method in which a preferred aerosol is identified may be followed by step S4. Similarly, steps S31 to S33 may be performed without subsequently performing step S34. In one case, steps S33 and S34 may be omitted, and the user may manually control the aerosol generation device 1 based on the candidate preferred aerosols presented in step S32.

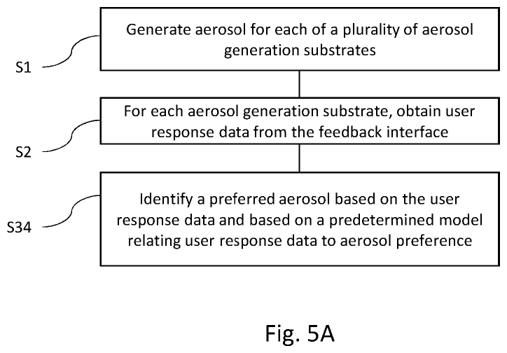

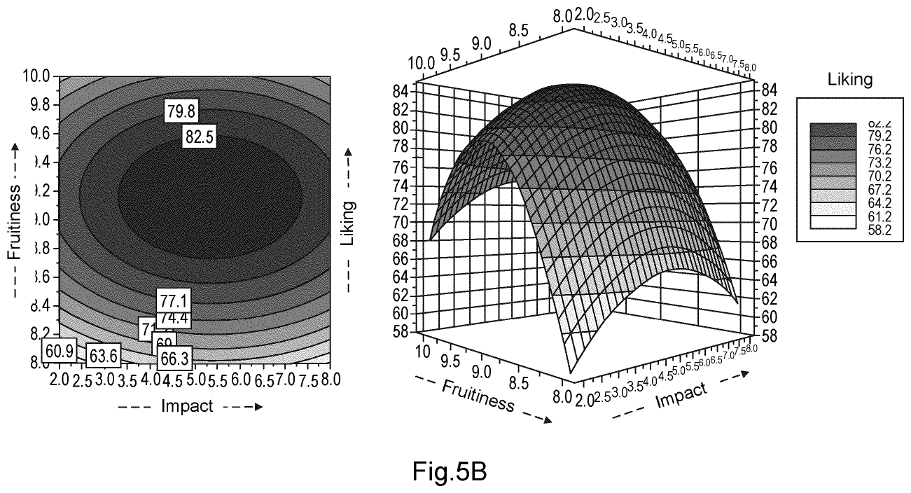

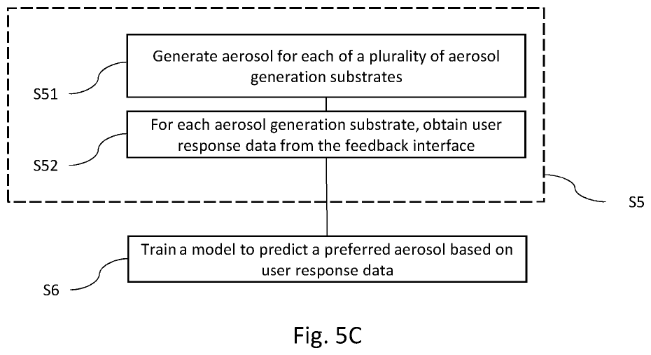

FIG. 5A is a schematic flow chart illustrating further optional features of the method shown in FIG. 3. This may be combined with the first modification shown in FIG. 4. Steps S1 and S2 are identical to the above-described method of FIG. 3, and will not be described again.

In the modified method of FIG. 5A, step S3 of FIG. 3 is replaced with an alternative step S34 in which the preference identification system identifies the preferred aerosol for the user based on the user response data obtained in step S2 and based on a predetermined model relating user response data to aerosol preference.

The model may also be extended to predict more than a preferred aerosol. For example, the model may be trained to determine a health characteristic of the user based on the user response data, and allowing or blocking use of an aerosol generation substrate, in the identification of a possible next aerosol, depending upon the user response data. In one example, if a response to a previous aerosol was had a high concentration of an active ingredient, an aerosol generation setting may be identified in which the quantity of an active ingredient is limited to below a threshold.

FIG. 5B depicts an example representation of a predetermined model that may be used in step S34. More specifically, FIG. 5B shows a 2D heat map and a 3D graph of voluntary response data, or “liking”, against a scaled “fruitiness” flavour characteristic and an impact characteristic (such as nicotine strength). The 2D heat map and 3D graph can only represent a part of the model, and the model will also describe the relationships between the involuntary response data such as heart rate and galvanic skin response and the voluntary response data. For example, the trained model may comprise a series of graphs, one or more tables, or more preferably a parametric definition of the model. By fitting the model to the user response data obtained in step S2 for an individual user, the preference identification system can identify a predicted peak “liking” point for the individual user, and identify an aerosol ng associated with the peak as a preferred aerosol.

FIG. 5C is a schematic flow chart illustrating a method for training the model for predicting the preferred aerosol for a user. This method may be performed by the preference identification system in communication with a plurality of aerosol generation devices using a test sample of initial training users of the device. Alternatively, the method of FIG. 5C may be continuously performed in order to improve the model based on user responses from all of, or a subset of, the users of the device when it is widely deployed.

Referring to FIG. 5C, at step S5, the preference identification system collects response data for each of a plurality of training users who participate in training of the model, from one or more aerosol generation devices. Steps S51 and S52 may be similar to steps S1 and S2 of FIG. 3.

The user response data is then collected together to train a model at step S6. Specifically, the model is trained to predict the preferred aerosol of a user based on user response data, the training being based on user response data for the plurality of training users. The trained model can then be used in step S34 of FIG. 5A to identify a preferred aerosol based on user response data at a specific aerosol generation device 1.

For example, the trained model may be provided as a predetermined model stored in the control circuitry 13 or the remote processing unit 5 to map between user response data and preferred aerosols. Thus, the method of FIG. 5C may be performed entirely separately from the method of FIG. 5A.

Step S6 may be performed using any machine learning technique. For example, the training may be based on Principle Component Analysis (PCA), General Linear Regression Models (GLM), or Partial Least Square (PLS) analysis.

Such machine learning can also be used to predict properties other than preferred aerosol. For example, machine learning can be used to reduce raw user response data to more general response characteristics, such as arousal and valence.

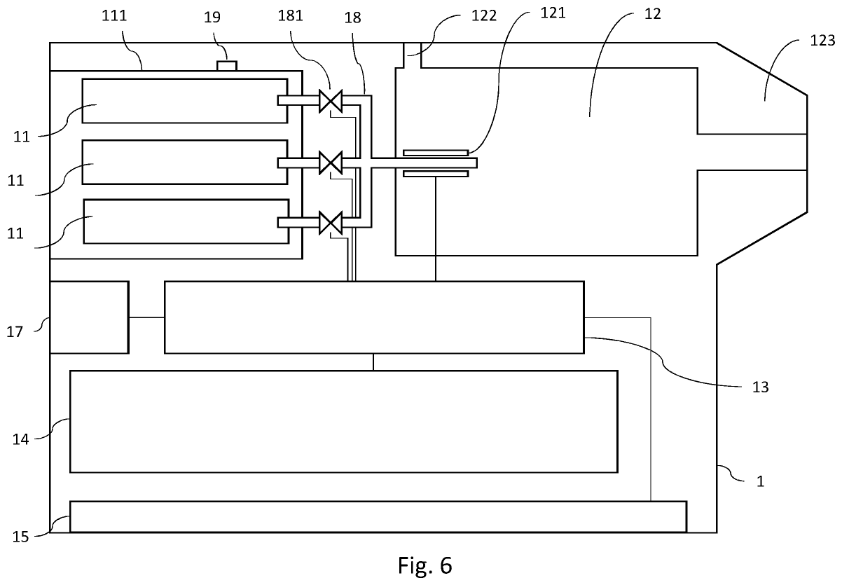

FIG. 6 schematically illustrates an alternative aerosol generation device for use in an aerosol generation system according to the invention.

In this embodiment, the aerosol generation substrate is a liquid aerosol substrate such as a volatile oil, and may contain tobacco or a tobacco extract such as nicotine.

The aerosol generation device 1 comprises a plurality of substrate storage chambers 11, an aerosol generation chamber 12 and control circuitry 13. The substrate storage chambers 11 each store an aerosol generation substrate, and the aerosol generation chamber 12 is configured to generate a respective aerosol component from each of the aerosol generation substrates and to mix the aerosol components to form the aerosol. The control circuitry 13 is configured to control the aerosol generation chamber 12 and to control supply of the aerosol generation substrates from the substrate storage chambers 11 to the aerosol generation chamber 12.

The aerosol generation substrate stored in each chamber 11 may be a different substrate that produces a different aerosol component. For example, each aerosol generation substrate may have a different flavour. Alternatively, two or more substrate storage chambers 11 may contain the same aerosol generation substrate.

Optionally, the substrate storage chambers 11 may be provided together in a removable cartridge 111, which can be used to replace the substrate storage chambers 11 after the stored aerosol generation substrate has been used. The removable cartridge 111 may be compared to the pack 2 of FIG. 1B. Alternatively, each of the substrate storage chambers 11 may be individually replaceable.

Although three substrate storage chambers 11 are shown in FIG. 6, any number of one, two or more substrate storage chambers 11 may be used.

Each aerosol generation substrate is conveyed from its substrate storage chamber 11 to the aerosol generation chamber 12 via a tube 18. The tube 18 may be a capillary tube such that the liquid aerosol generation substrates flow along the tube 18 without requiring a driving pressure. Alternatively, the tube 18 may comprise one or more pumps for driving each aerosol generation substrate along the tube 18.

The tube 18 comprises a valve 181 for controlling flow of the aerosol generation substrate from the substrate storage chamber 11 to the aerosol generation chamber 12. Each valve 181 can be controlled to be anywhere between fully closed and fully open according to a required amount of the respective aerosol generation substrate for generating a corresponding aerosol component.

The tube 18 acts as a substrate mixing element configured to mix the aerosol generation substrates and to supply the mixed aerosol generation substrates to the aerosol generation chamber 12. More specifically, separate branches of the tube 18 connect to each substrate storage chamber 11 and merge after the valves 181 such that the aerosol generation substrates are mixed together before reaching the aerosol generation chamber 12.

The aerosol generation chamber comprises a heating element 121 which is configured to generate the respective aerosol component by heating each of the aerosol generation substrates. In this embodiment, heating element is a resistive coil wrapped around the tube 18. The heating element could alternatively, for example, be a planar film heater.

Due to the branching of tube 18, the aerosol generation substrates are already mixed before reaching the heating element 121, and so the aerosol components are generated and mixed simultaneously.

Once the aerosol components have been mixed to produce an aerosol, a user of the aerosol generation device can draw the aerosol out of the device by inhaling to draw air into the aerosol generation chamber 12 through an inlet 122 and out of the aerosol generation chamber 12 through a mouthpiece 123.

The aerosol generation device of FIG. 6 provides an aerosol that can comprise a mixture of components. The control circuitry 13 is configured to adjust a ratio of these aerosol components generated by the aerosol generation chamber 12. More specifically, the control circuitry 13 is configured to control the heating element 121 and is configured to control the valves 181 to set a ratio of the aerosol generation substrates in a combined liquid that reaches the heating element 121, thereby setting a ratio of the aerosol components produced by heating the combined liquid.

The control circuitry 13 may control the ratio(s) of the aerosol generation substrates based on a preferred aerosol identified using one of the above-described methods. In such embodiments, the preferred aerosol may be indicated numerically using relative quantities of a set of known possible aerosol components that are included in the preferred aerosol.

Additionally, the control circuitry 13 may generate an aerosol for each of a random subset of possible aerosols, by controlling the ratio(s) of the aerosol generation substrates that are mixed together. By introducing randomness into the aerosol generation, a more complete exploration of possible aerosols can be achieved.

Although the example of FIG. 6 is described for liquid substrates stored in chambers 11, the concept of mixing components could be applied to solid substrates, for example by mixing powdered substrate components.

In either of the embodiments shown in FIGS. 1 and 6, the aerosol generation device may further comprise a substrate identification means 19, in order to identify the substrate 11 that is being used in the aerosol generation device. For example, the aerosol generation device of FIG. 1 may be configured to read a bar code on wrapper 111. Similarly, the aerosol generation device of FIG. 6 may be configured to read an identifier chip from removable cartridge 111. The identified substrate may then be provided to the feedback interface or the preference identification system for use in identifying the preferred aerosol. By automatically identifying the substrate, the preference identification system can associate user response data with specific aerosol generation substrates, without requiring a manual indication of the aerosol generation substrate that is being used, thereby improving accuracy and convenience of the aerosol generation system.

### Example Usage

We now provide a specific example of how an aerosol generation system as described above may be used.

First, after being activated for a first time, the aerosol generation system performs an initial learning phase.

The initial learning phase comprises providing 15 different aerosol generation sessions each generating a different aerosol. During each session, the aerosol generation device monitors involuntary response data comprising heart rate, blood pressure, skin temperature and galvanic skin response, and also monitors aerosol inhalation. After each session, the user provides voluntary response data by rating the aerosol in an application on their smart phone 4. The aerosol generation device communicates user response data comprising the involuntary response data, inhalation data and the voluntary response data to the remote processing unit 6 which functions as a preference identification system.

The remote processing unit receives and analyses the data to determine a preference pattern.

The remote processing unit 6 sends the preference pattern to an application on the smart phone 4, which provides feedback to the user in the form of:
a contour plot indicating preferences on a sensory map of the different aerosol generation substrates,
a bar chart indicating how well or badly a user reacted to each different aerosol generation substrate or each other factor controlling the generated aerosol,
an automatically generated text summary of the user's preferences, and
feedback on how the sessions affected the user's relaxation or arousal.

Once the above initial learning phase has been completed, the remote processing unit 5 can provide suggestions for new aerosols that the user is likely to enjoy. The suggestions may be provided in the application on the smart phone 3. For example, the suggestions may be displayed on a further sensory map.

The suggestions may be generated based on a machine learning model as described above. Furthermore, the response data may be analysed to identify one or more user parameters for suggesting aerosols. For example, a Kano model may be used to identify “must-haves”, “performance drivers” or “attractors” in order to classify potential aerosols and generate suggested aerosols that are likely to be attractive to a specific user.

When the user wishes to select a suggested aerosol, they indicate this to the aerosol generation device 1. In a preferred example, the application on the smart phone 3 is configured to receive the selection (for example as a selection on the sensory map) and communicate the selection to the aerosol generation device 1. Alternatively, the user may indicate the selection by controlling the aerosol at an interface of the aerosol generation device 1, or by manually inserting a recommended aerosol generation substrate into the aerosol generation device 1.

Once an aerosol has been selected, the aerosol generation device provides an aerosol generation session generating the selected aerosol, and further user responses are measured based on the selected aerosol in order to provide further feedback and suggestions.

In the above-described methods, preferred aerosols are identified based on user response data including voluntary response data, inhalation data, and involuntary response data. Other data may be also be used to assess user preferences. For example, environmental parameters such as date, time, day of week and location may be used to identify patterns in user preferences and habits.

In both of the embodiments of FIGS. 1 and 6, each different aerosol may comprise generating an aerosol from a different aerosol generation substrate, and/or generating an aerosol at a different strength of aerosol generation by the heating element 121.

The different aerosols preferably comprise a range of predetermined aerosols that are known to be preferred by individual users. Alternatively, the aerosol preferences may be constrained to a large but finite number of possible aerosol preferences.

For example, if six aerosol generation substrates (five flavours and one nicotine component) are mixed, each substrate is included in one of three levels (high, medium, low), and a strength of aerosol generation by the heating element 121 is also high, medium or low, then there are 3\hat{ }7=2187 possible aerosols which can be generated.

1. An aerosol generation system comprising:
an aerosol generation device configured to receive each of a plurality of aerosol generation substrates and to generate a respective aerosol to be inhaled by a user;
a feedback interface for receiving user response data of a user in reaction to inhaling the generated aerosol; and
a preference identification system configured to:
for each of the plurality of aerosol generation substrates, obtain the user response data from the feedback interface; and
identify a preferred aerosol for the user based on the user response data.

2. The aerosol generation system according to claim 1, wherein the preference identification system is configured to:
obtain initial user response data from the feedback interface for each of a sample plurality of aerosol generation substrates, the sample plurality of aerosol generation substrates being provided in a pack; and
identify an initial preferred aerosol for the user based on the initial user response data.

3. The aerosol generation system according to claim 2, wherein the respective aerosols generated from the sample plurality of aerosol generation substrates are distributed across a sensory range of different compositions.

4. The aerosol generation system according to claim 3, wherein the sensory range comprises a range of strengths of each of one or more aerosol components.

5. The aerosol generation system according to claim 2, wherein the preference identification system is further configured to:
obtain subsequent user response data from the feedback interface for one or more subsequent aerosol generation substrates;
identify an updated preferred aerosol for the user based on the subsequent user response data and the initial preferred aerosol.

6. The aerosol generation system according to claim 1, wherein the preference identification system is configured to:
identify a plurality of candidate preferred aerosols for the user based on the user response data; and
present the plurality of candidate preferred aerosols in a user interface.

7. The aerosol generation system according to claim 1, wherein the aerosol generation device is configured to receive at least two of the plurality of aerosol generation substrates simultaneously, and to control which of the received aerosol generation substrates is used to generate an aerosol.

8. The aerosol generation system according to claim 7, wherein the aerosol generation device is configured to receive an indication of the preferred aerosol, and to control which of the received aerosol generation substrates is used to generate an aerosol, based on the preferred aerosol.

9. The aerosol generation system according to claim 7, wherein the aerosol generation device comprises an aerosol generation chamber and a substrate mixing element configured to mix a plurality of the simultaneously-received aerosol generation substrates and supply a mixed aerosol generation substrate to a heating chamber.

10. The aerosol generation system according to claim 1, wherein the preference identification system is configured to identify the preferred aerosol based on the user response data and based on a predetermined model relating user response data to aerosol preference.

11. The aerosol generation system according to claim 10, wherein the preference identification system is configured to:
obtain user response data for each of a plurality of training users; and
train the model to predict a preferred aerosol based on user response data for the plurality of training users.

12. The aerosol generation system according to claim 1, wherein the feedback interface comprises a sensor for measuring an involuntary response of the user, and the user response data comprises an indication of the involuntary response.

13. The aerosol generation system according to claim 12, wherein the sensor is configured to measure a heart rate, a blood pressure, a skin temperature or a galvanic skin response.

14. The aerosol generation system according to claim 12, wherein the sensor is arranged on an exterior surface of a housing of the aerosol generation device.

15. The aerosol generation system according to claim 1, wherein the feedback interface comprises a voluntary response interface for receiving a voluntary response from the user in reaction to inhaling the generated aerosol, and the user response data comprises an indication of the voluntary response.

16. The aerosol generation system according to claim 1, wherein the feedback interface comprises an air flow sensor arranged to measure aerosol inhalation at the aerosol generation device when the user is inhaling the generated aerosol, and the user response data comprises an indication of the aerosol inhalation.

17. The aerosol generation system according to claim 1, wherein the aerosol generation device comprises a substrate identification readable material for identifying a received aerosol generation substrate, and the user response data comprises an indication of the received aerosol generation substrate.

18. (canceled)

19. A kit, comprising:
a pack comprising:
a sample plurality of aerosol generation substrates, each for generating a different respective aerosol in an aerosol generation system, wherein the different respective aerosols are distributed across a sensory range of different compositions such that a preferred aerosol can be identified based on user response data obtained in reaction to inhaling each of the aerosols; and
and an aerosol generation system comprising:
an aerosol generation device configured to receive each of the plurality of aerosol generation substrates and to generate the respective aerosol to be inhaled by a user;
a feedback interface for receiving user response data of a user in reaction to inhaling the generated aerosol; and
a preference identification system configured to:
for each of the plurality of aerosol generation substrates, obtain the user response data from the feedback interface; and
identify a preferred aerosol for the user based on the user response data.

20. A method of controlling an aerosol generation system, the aerosol generation system comprising:
an aerosol generation device configured to receive an aerosol generation substrate and to generate a respective aerosol to be inhaled by a user; and
a feedback interface for receiving user response data of the user in reaction to inhaling the generated aerosol,
the method comprising:
for each of a plurality of aerosol generation substrates, using the aerosol generation device to generate a respective aerosol, and obtaining user response data from the feedback interface; and
identifying a preferred aerosol for the user based on the user response data.
